(19) United States
(12) Patent Application Publication
(10) Pub. No.: US 2017/0009210 A1
(43) Pub. Date: Jan. 12, 2017

(54) GUIDED DIFFERENTIATION OF INDUCED PLURIPOTENT STEM CELLS

(71) Applicant: MAYO FOUNDATION FOR MEDICAL EDUCATION AND RESEARCH, Rochester, MN (US)

(72) Inventors: Yasuhiro Ikeda, Rochester, MN (US); Moustafa M.A. El Khatib, Rochester, MN (US)

(73) Assignee: Mayo Foundation for Medical Education and Research, Rochester, MN (US)

(21) Appl. No.: 15/115,784

(22) PCT Filed: Feb. 4, 2015
(86) PCT No.: PCT/US2015/014377

§ 371 Date: Aug. 1, 2016

Related U.S. Application Data
(60) Provisional application No. 61/935,944, filed on Feb. 5, 2014.

(51) Int. Cl. C12N 5/071; C12N 5/074

(52) U.S. Cl. CPC C12N 5/0676; C12N 5/0696; C12N 2506/094; C12N 2506/45; C12N 2501/734; C12N 2510/00; C12N 2501/16; C12N 2501/335; C12N 2501/603; C12N 2501/602; C12N 2501/604; C12N 2501/606; C12N 2501/999

(57) ABSTRACT

This document provides methods and materials related to making and using differentiated induced pluripotent stem cells. For example, methods and materials for making differentiated induced pluripotent stem cells (e.g., insulin-producing cells) that do not form cancer cells within a mammal (e.g., a human), cells that underwent guided differentiation from induced pluripotent stem cells, compositions containing cells that underwent guided differentiation from induced pluripotent stem cells, and methods for using cells that underwent guided differentiation from induced pluripotent stem cells (e.g., methods for using such cells to treat diabetes or to repair cardiovascular tissue) are provided.

### CROSS-REFERENCE TO RELATED APPLICATIONS

This application claims the benefit of U.S. Provisional Application Ser. No. 61/935,944, filed Feb. 5, 2014. The disclosure of the prior application is considered part of (and is incorporated by reference in) the disclosure of this application.

### BACKGROUND

1. Technical Field

This document relates to methods and materials involved in making and using differentiated induced pluripotent stem cells. For example, this document provides methods and materials for making differentiated induced pluripotent stem cells (e.g., insulin-producing cells) that do not form cancer cells within a mammal (e.g., a human).

2. Background Information

Stem cells are characterized by the ability of self-renewal and differentiation into a diverse range of cell types. The two broad types of mammalian stem cells are embryonic stem (ES) cells and adult stem cells. Adult stem cells or progenitor cells replenish specialized cells to repair or maintain regenerative organs. Most adult stem cells are lineage-restricted and generally referred to by their tissue origin, such as adipose-derived stem cells. ES cell lines are derived from the epiblast tissue of the inner cell mass of a blastocyst or early morula stage embryos. ES cells are pluripotent and give rise to derivatives of the three germinal layers, i.e., the ectoderm, endoderm, and mesoderm.

### SUMMARY

This document provides methods and materials related to making and using differentiated induced pluripotent stem cells. For example, this document provides methods and materials for making differentiated induced pluripotent stem cells (e.g., insulin-producing cells) that do not form cancer cells within a mammal (e.g., a human). This document also provides cells that underwent guided differentiation from induced pluripotent stem cells, compositions containing cells that underwent guided differentiation from induced pluripotent stem cells, and methods for using cells that underwent guided differentiation from induced pluripotent stem cells (e.g., methods for using such cells to treat diabetes or to repair cardiovascular tissue).

As described herein, induced pluripotent stem cells can be guided to differentiate into cells that, when implanted into a mammal, do not form cancer cells (e.g., teratomas or teratocarcinoma). For example, induced pluripotent stem cells can be obtained from somatic cells using one or more non-integrating vectors designed to express polypeptides that reprogram the somatic cells to create induced pluripotent stem cells. Examples of such non-integrating vectors include Sendai viral vectors. In addition, at one or more time points during the differentiation process of such induced pluripotent stem cells into more specialized cells, the cells can be exposed to enzymatic treatment. For example, induced pluripotent stem cells produced using one or more non-integrating vectors can be exposed to (a) factors designed to differentiate the induced pluripotent stem cells into more specialized cells and (b) a protease (e.g., a serine protease such as trypsin). Using non-integrating vectors to produce the induced pluripotent stem cells and using enzymatic treatment during the guided differentiation process of the induced pluripotent stem cells to form more specialized cells can produce specialized cells that, when implanted into a mammal, do not form cancer cells (e.g., teratomas or teratocarcinoma).

In general, one aspect of this document features a population of differentiated cells obtained from induced pluripotent stem cells. The cells of the population lack integrated viral nucleic acid encoding a stemness factor, wherein implantation of the population of differentiated cells into a mammal does not result in cancer cell formation, and wherein the cells were obtained using a culture comprising an enzyme. The cells can be human cells. The cells can lack exogenous nucleic acid. The enzyme can be trypsin.

In another aspect, this document features a method for obtaining a population of differentiated cells obtained from induced pluripotent stem cells. The method comprises, or consists essentially of, (a) exposing somatic cells to one or more non-integrating viral vectors that direct the expression of one or more sternness factors to produce induced pluripotent stem cells from the somatic cells, and (b) exposing the induced pluripotent stem cells to one or more differentiation factors in the presence of an enzyme to produce a population of cells more specialized than the induced pluripotent stem cells. The somatic cells can be keratinocytes. The one or more non-integrating viral vector can be Sendai viral vectors. The one or more sternness factors can be an Oct3/4 polypeptide, a Sox2 polypeptide, a Klf4 polypeptide, or a c-Myc polypeptide. The differentiation factors can be activin A, wnt3a, KAAD-cyclopamine, retinoic acid, indolactam V, IGF1, HGF, DAPT, BMP4, exendine 4, TGF-beta/BMP inhibitors, Sonic hedgehog inhibitors, PKC activators, GLP1, or a combination thereof. The enzyme can be trypsin.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a diagram of pancreatic differentiation of lentivirally reprogrammed iPSCs. During the guided differentiation steps and before transplantation, the cells were dissociated by non-enzymatic dissociation buffer, Versene™, obtained from Invitrogen.

FIG. 2 contains photographs of a mouse 6-8 weeks post-transplantation with pancreatic differentiation of lentivirally reprogrammed iPSCs treated with Versene™ and a teratoma removed from such a mouse. Transplantation of iPSC-derived pancreatic endoderm cells resulted in teratoma formation. FIG. 2 also contains a table demonstrating that palpable growths were detected in nearly 100% of mice that received cells from iPSCs generated using lentiviral vectors.

FIG. 3 contains photographs demonstrating the frequent recurrent and metastatic nature of tumors in recipient mice after teratoma removal by nephrectomy.

FIG. 4 contains a schematic diagram of replication-deficient AF Sendai vectors along with photographs of cell staining results.

FIG. 5 contains staining results obtained using transgene-free iPSCs from elderly patients with diabetes using non-integrating Sendai virus vectors. Similarities were observed in global gene express profiles between iPSCs made with lentiviral vectors as compared to those made with Sendai viral vectors.

FIG. 6 is a diagram of pancreatic differentiation of iPSCs obtained using non-integrating viral vectors (e.g., Sendai viral vectors). During the guided differentiation and before transplantation, Sendai-iPSC-derived progeny cells were dissociated using trypsin. This approach allowed for teratoma-free transplantation of iPSC-derived progeny. Use of lentiviral vectors along with enzymatic dissociation steps resulted in teratoma formation. These results demonstrate that teratoma-free iPSC transplantation requires transgene-free iPSCs (such as Sendai virus-iPSCs).

FIG. 7 contains results demonstrating that use of transgene-free iPSCs and enzymatic dissociation allowed regeneration of human islets without teratoma formation.

FIG. 8 contains results demonstrating that enzymatic dissociation is necessary for teratoma-free transplantation. During the guided differentiation and before transplantation, iPSC-derived progeny cells were dissociated by non-enzymatic Versine™. Use of Sendai-iPSC line, without enzymatic dissociation steps, for transplantation resulted in teratoma formation. Thus, teratoma-free iPSC transplantation requires enzymatic dissociation.

FIG. 9 contains results demonstrating that lentiviral integration of c-Myc gene plays a role in teratoma formation after transplantation of iPSC progeny. To understand the role of reprogramming lentivector integration on teratoma formation, Sendai-iPSC RD16-A line was further infected with lentivectors, and used for transplantation upon guided pancreatic differentiation with enzymatic dissociation. During the guided differentiation and before transplantation, iPSC-derived progeny cells were dissociated by trypsin. Lentiviral integration of one of the four reprogramming factors, cMYC, was sufficient to prevent teratoma-free transplantation of Sendai-iPSC line, RD16-A.

### DETAILED DESCRIPTION

This document provides methods and materials related to making and using differentiated induced pluripotent stem cells. For example, this document provides methods and materials for making differentiated induced pluripotent stem cells (e.g., insulin-producing cells) that do not form cancer cells within a mammal (e.g., a human). This document also provides cells that underwent guided differentiation from induced pluripotent stem cells, compositions containing cells that underwent guided differentiation from induced pluripotent stem cells, and methods for using cells that underwent guided differentiation from induced pluripotent stem cells (e.g., methods for using such cells to treat diabetes or to repair cardiovascular tissue).

As described herein, the methods and materials provided herein can be used to guide the differentiation of induced pluripotent stem cells into more specialized cells that, when implanted into a mammal, do not form cancer cells (e.g., teratomas or teratocarcinoma). Any appropriate induced pluripotent stem cell population can be used to make these more specialized cells provided that the induced pluripotent stem cells do not contain a transgene or viral vector encoding a c-Myc polypeptide. In some cases, induced pluripotent stem cells produced using non-integrating viral vectors designed to express reprograming factors (e.g., an Oct3/4 polypeptide, a Sox family polypeptide such as a Sox2 polypeptide, a Klf family polypeptide such as a Klf4 polypeptide, a Myc family polypeptide such as a c-Myc polypeptide, a Nanog polypeptide, a Lin28 polypeptide, or a combination thereof) can be used to make these more specialized cells. Examples of non-integrating viral vectors that can be used to produce induced pluripotent stem cells from somatic cells include, without limitation, Sendai viral vectors, measles viral vectors, parainfluenza viral vectors, adenoviral vectors, adeno-associated virus vectors, and non-integrating lentiviral vectors with mutated integrase. In some cases, the techniques described elsewhere (e.g., Fusaki et al., Proc. Jpn. Acad., Ser. B, 85:348-362 (2009)) can be used to make induced pluripotent stem cells from somatic cells.

The polypeptides used to induce the formation of induced pluripotent stem cell can include any combination of Oct3/4 polypeptides, Sox family polypeptides (e.g., Sox2 polypeptides), Klf family of polypeptides (e.g., Klf4 polypeptides), Myc family polypeptides (e.g., c-Myc), Nanog polypeptides, and Lin28 polypeptides. For example, nucleic acid vectors designed to express Oct3/4, Sox2, Klf4, and c-Myc polypeptides can be used to obtain induced pluripotent stem cells. In some cases, Oct3/4, Sox2, Klf4, and c-Myc polypeptides can be directly delivered into target cells to obtain induced pluripotent stem cells using a polypeptide transfection method (e.g., liposome or electroporation). In one embodiment, nucleic acid vectors (e.g., non-integrating vectors) designed to express Oct3/4, Sox2, and Klf4 polypeptides, and not a c-Myc polypeptide, can be used to obtain induced pluripotent stem cells. In some cases, Oct3/4, Sox2, and Klf4 polypeptides can be directly delivered into target cells to obtain induced pluripotent stem cells using a polypeptide transfection method. An Oct3/4 polypeptide can have the amino acid sequence set forth in GenBank® Accession Numbers BC117435 (e.g., GI No. 109659099). An Sox2 polypeptide can have the amino acid sequence set forth in GenBank® Accession Numbers BC013923 (e.g., GI No. 33869633). A Klf4 polypeptide can have the amino acid sequence set forth in GenBank® Accession Numbers BCO29923 (e.g., GI No. 20987475). A c-Myc polypeptide can have the amino acid sequence set forth in GenBank® Accession Numbers BC000141 (e.g., GI No. 12652778). A Nanog polypeptide can have the amino acid sequence set forth in GenBank® Accession Numbers BC099704.1 (e.g., GI No. 71043476). A Lin28 polypeptide can have the amino acid sequence set forth in GenBank® Accession Numbers BCO28566 (e.g., GI No. 33872076).

Any appropriate cell type can be used to obtain induced pluripotent stem cells. For example, skin, lung, heart, liver, blood, kidney, or muscle cells can be used to obtain induced pluripotent stem cells. Such cells can be obtained from any type of mammal including, without limitation, humans, mice, rats, dogs, cats, cows, pigs, or monkeys. In some cases, induced pluripotent stem cells can be produced from keratinocytes (e.g., keratinocytes from health mammals such as healthy humans, keratinocytes from mammals with type 1 diabetes such as human type 1 diabetics, or keratinocytes from mammals with type 2 diabetes such as human type 2 diabetics), peripheral blood, hematopoietic stem cells, jejunum mucosal myofibroblasts, dermal fibroblasts, cardiac fibroblasts, bone marrow cells, or mesenchymal stem cells. In addition, any stage of the mammal can be used, including mammals at the embryo, neonate, newborn, or adult stage. For example, fibroblasts obtained from an adult human patient can be used to obtain induced pluripotent stem cells. Such induced pluripotent stem cells can be used to treat that same human patient (or to treat a different human) or can be used to create differentiated cells that can be used to treat that same human patient (or a different human). For example, somatic cells from a human patient can be treated as described herein to obtain induced pluripotent stem cells. The obtained induced pluripotent stem cells can be differentiated into other cell types (e.g., insulin-producing cells or cardiomyocytes) that can be implanted into that same human patient.

Any appropriate method can be used to introduce nucleic acid (e.g., nucleic acid encoding polypeptides designed to induce pluripotent stem cells from cells) into a cell. For example, nucleic acid encoding polypeptides (e.g., Oct3/4, Sox2, Klf4, and c-Myc polypeptides) designed to induce pluripotent stem cells from other cells (e.g., non-embryonic stem cells) can be transferred to the cells using recombinant viruses that can infect cells, or liposomes or other non-viral methods such as electroporation, microinjection, transposons, phage integrases, or calcium phosphate precipitation, that are capable of delivering nucleic acids to cells. The exogenous nucleic acid that is delivered typically is part of a vector in which a regulatory element such as a promoter is operably linked to the nucleic acid of interest. The promoter can be constitutive or inducible. Non-limiting examples of constitutive promoters include cytomegalovirus (CMV) promoter and the Rous sarcoma virus promoter. As used herein, “inducible” refers to both up-regulation and down regulation. An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent such as a protein, metabolite, growth regulator, phenolic compound, or a physiological stress imposed directly by, for example heat, or indirectly through the action of a pathogen or disease agent such as a virus.

Additional regulatory elements that may be useful in vectors, include, but are not limited to, polyadenylation sequences, translation control sequences (e.g., an internal ribosome entry segment, IRES), enhancers, or introns. Such elements may not be necessary, although they can increase expression by affecting transcription, stability of the mRNA, translational efficiency, or the like. Such elements can be included in a nucleic acid construct as desired to obtain optimal expression of the nucleic acids in the cells. Sufficient expression, however, can sometimes be obtained without such additional elements.

Vectors also can include other elements. For example, a vector can include a nucleic acid that encodes a signal peptide such that the encoded polypeptide is directed to a particular cellular location (e.g., the cell surface) or a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture.

Any appropriate non-viral vectors can be used to introduce sternness-related factors, such as Oct3/4, Klf4, Sox2, and c-Myc. Examples of non-viral vectors include, without limitation, vectors based on plasmid DNA or RNA, retroelement, transposon, and episomal vectors. Non-viral vectors can be delivered to cells via liposomes, which are artificial membrane vesicles. The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Transduction efficiency of liposomes can be increased by using dioleoylphosphatidylethanolamine during transduction. See, Felgner et al., J. Biol. Chem., 269:2550-2561 (1994). High efficiency liposomes are commercially available. See, for example, SuperFect® from Qiagen (Valencia, Calif.).

In some cases, other reprogramming strategies that do not involve sternness-related factors can be used to generate induced pluripotent stem cells. For example, the introduction of micro RNAs such as miR302 and miR367 or the treatment of somatic cells with combinations of reprogramming small molecules can be used to generate induced pluripotent stem cells.

In some cases, induced pluripotent stem cells can be obtained using culture conditions that do not involve the use of serum or feeder cells. For example, cells obtained from a human can be provided nucleic acid encoding human Oct3/4, Sox2, Klf4, and c-Myc polypeptides and cultured using media lacking serum (e.g., human or non-human serum) and lacking feeder cells (e.g., human or non-human feeder cells).

Once the polypeptides are expressed and induced pluripotent stem cells are obtained, the induced pluripotent stem cells can be maintained in culture such that the induced pluripotent stem cells are devoid of the exogenous nucleic acid.

Once transgene-free induced pluripotent stem cells are obtained, the cells can be exposed to factors designed to guide differentiation of those induced pluripotent stem cells into more specialized cells. For example, when producing insulin-secreting cells, the induced pluripotent stem cells can be exposed to activin A, wnt3a, KAAD-cyclopamine, retinoic acid, indolactam V, IGF1, HGF, DAPT, BMP4, exendine 4, TGF-beta/BMP inhibitors, Sonic hedgehog inhibitors, PKC activators, and GLP1. When producing cardiomyocytes, the induced pluripotent stem cells can be exposed to activin A, BMP4, bFGF, extracellular matrix, and B27 supplement. In some cases, the techniques described elsewhere (e.g., Karakikes et al., Stem Cells Translational Medicine, 3:18-31 (2014)) can be used to produce cardiomyocytes from induced pluripotent stem cells. When producing hepatocytes, the induced pluripotent stem cells can be exposed to activin A, bFGF, retinoic acid, HGF, dexamethasone, nicotinamide, DMSO, and B27 supplement. In some cases, the techniques described elsewhere (e.g., Yu et al., Stem Cell Research, 9:196-207 (2012)) can be used to produce hepatocytes from induced pluripotent stem cells. When producing dopaminogenic neuronal cells, the induced pluripotent stem cells can be exposed to B27 supplement, N2 supplement, Matrigel, gelatin, fibronectin, mouse Laminin, poly-L-ornithine hydrobromide, Sonic hedgehog SHH-C25II substitution, Noggin, FGF8, BDNF, GDNF, Wnt3a, BMP4, NGF, NT-3, CNTF, Neuregulin, EGF, Dickkopf-1, TGFb-3, dcAMP, ascorbic acid, retinoic acid, SU5402, isobutylxanthine, dexamethasone, beta-glycerol phosphate, SB431542, y-27632 dihydrochloride, and LDN193189. In some cases, the techniques described elsewhere (e.g., Jiang et al., Nature Communications, 3:668 (2012)) can be used to produce dopaminergic neurons from induced pluripotent stem cells.

At one or more time points during the guided differentiation process, the cells can be exposed to an enzyme having the ability to kill residual human pluripotent stem cells. In some cases, the enzyme can be a protease. For example, the enzyme can be a serine protease such as trypsin. For elimination of residual human pluripotent stem cells, differentiating iPSC-derived progeny cells can be enzymatically dissociated using, for example, a recombinant trypsin/EDTA solution for 3 to 60 minutes (e.g., about 10 minutes) at 30° C. to 40° C. (e.g., about 37° C.). Trypsin can be neutralized with a defined trypsin inhibitor or a serum-containing medium. Dissociated cells can then be centrifuged, and cell pellets can be re-suspended in a serum-free medium. After a second centrifugation step, the iPSC progeny cell pellets can be resuspended in an appropriate differentiation medium and seeded in a matrix-coated or uncoated culture plate for further differentiation.

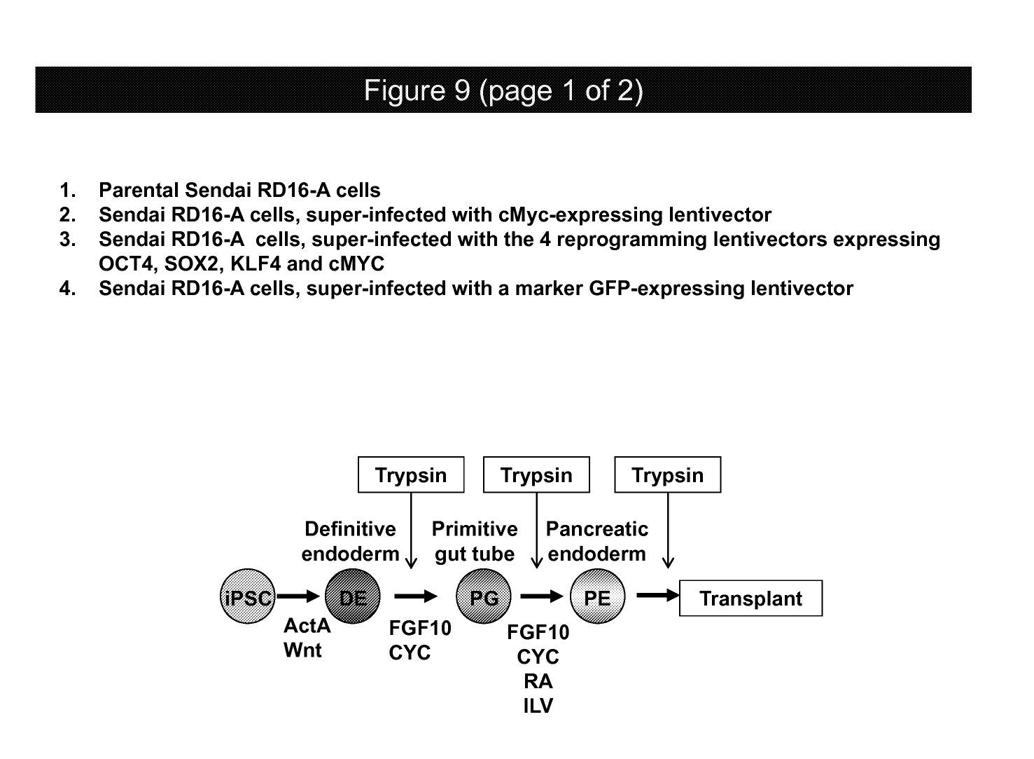

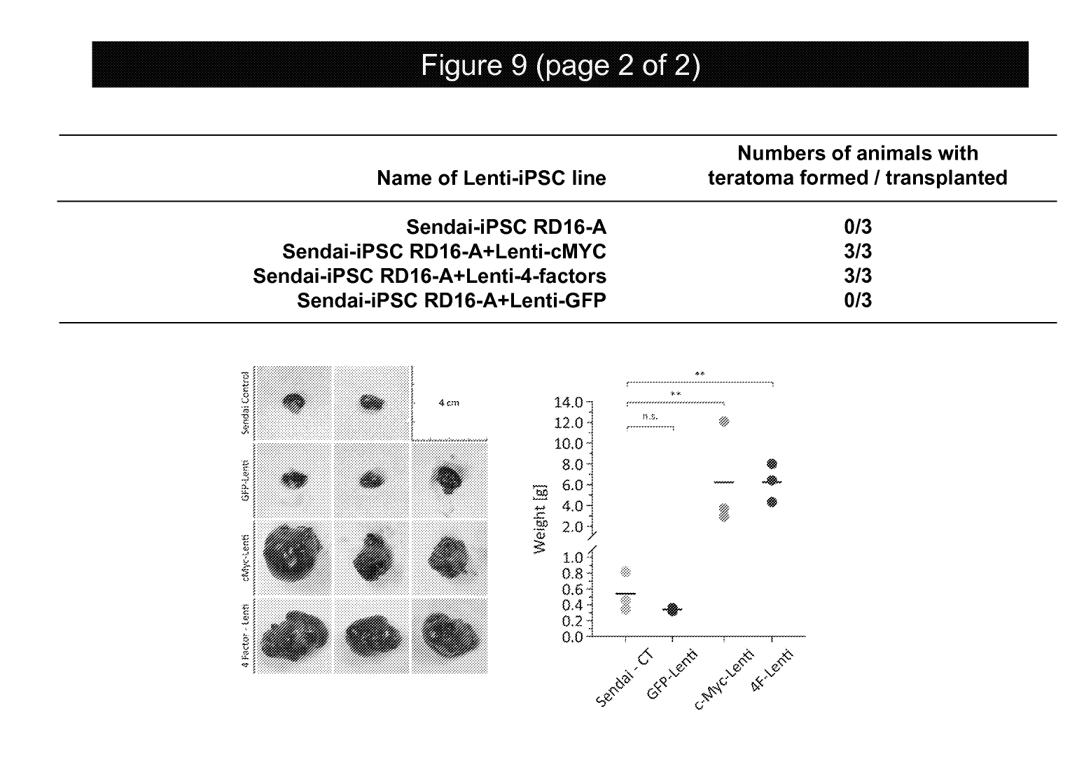

For verification of teratoma-free transplantation, iPSC-derived cells at the final stage of a step-wise guided differentiation can be enzymatically dissociated with a recombinant trypsin/EDTA solution for 1 to 30 minutes (e.g., 5 minutes) at 30° C. to 40° C. (e.g., about 37° C.). Trypsin can be neutralized with a defined trypsin inhibitor or a serum-containing medium. Dissociated cells can then be centrifuged, and cell pellets can be re-suspended in a serum-free medium. After a second centrifugation step, iPSC-differentiated cell pellets can be resuspended in an appropriate medium or saline for transplantation and kept on ice. Immuno-compromised SCID-beige mice can be used for transplantation. Mice can be anesthetized, and the kidney can be externalized for iPS progeny cell transplantation under the kidney capsule. A small incision can be made in the kidney capsule, and a blunt needle can be used to create a pocket under the kidney capsule. Following transplantation of iPSC progeny cells into the pocket, the kidney can be placed back into the abdomen, and the incision can be closed with vicryl suture. Mice can be monitored for up to 12 weeks for teratoma formation. Mice can be sacrificed for harvesting normal and iPSC progeny-transplanted kidneys. At about 12 weeks, teratoma-free transplants can result in no notable outgrowth from the renal capsule sites. In mice with teratoma, noticeable iPSC progeny outgrowth can be seen as early as 4 weeks after transplantation (see, e.g., FIG. 9).

The invention will be further described in the following examples, which do not limit the scope of the invention described in the claims.

### EXAMPLES

### Example 1

### Generating Differentiated Cells That Do Not Form Cancer Cells in a Mammal From iPSCs

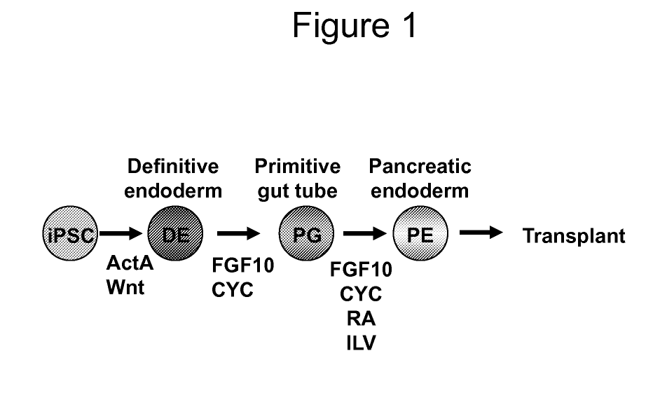

Schematic diagram of the differentiation process is shown as FIG. 1. iPSC clones were differentiated into insulin-producing endocrine cells through definitive endoderm, primitive gut tube, posterior foregut, and pancreatic progenitor stages.

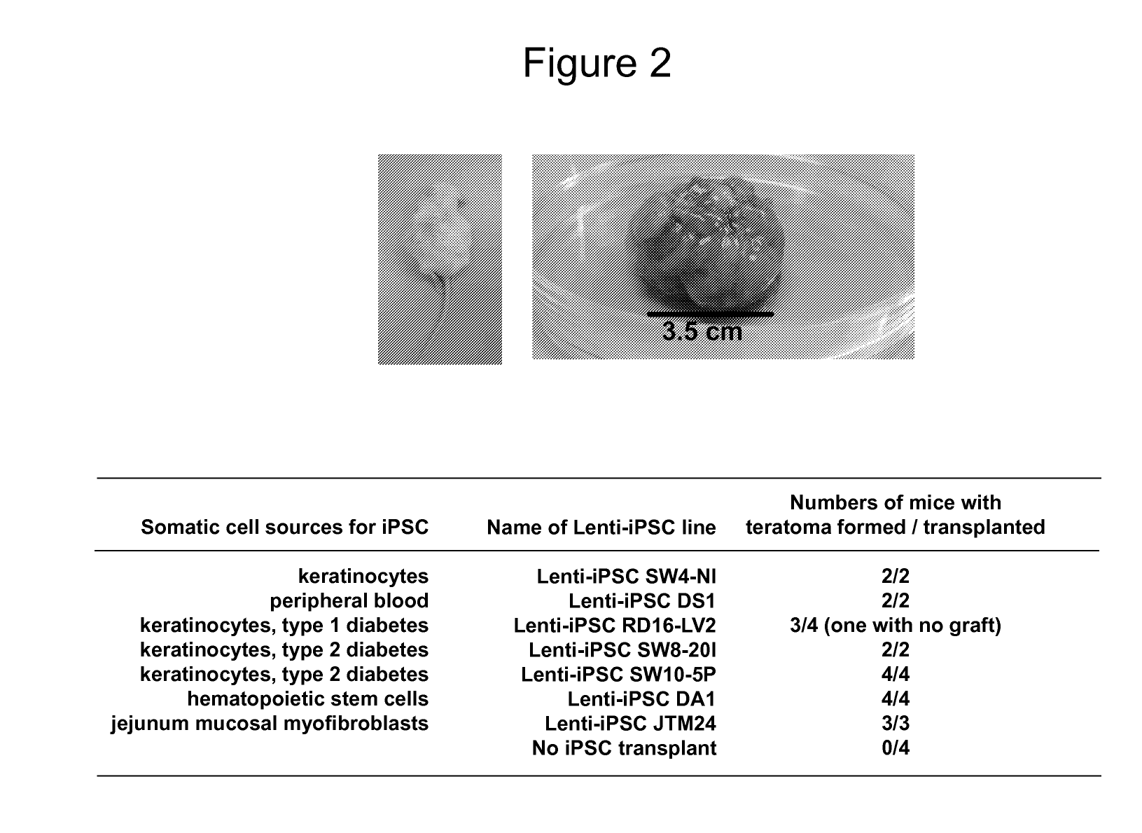

FIG. 2 demonstrates the consistent teratoma formation upon transplantation of pancreatic endoderm cells, which were derived from lentivector-reprogrammed iPSCs. Various iPSC lines from keratinocytes (Lenti-iPSC SW4-NI, Lenti-iPSC RD16-LV2, Lenti-iPSC SW8-20I, Lenti-iPSC SW10-5P), from peripheral blood (Lenti-iPSC DS1), from hematopoietic stem cells (Lenti-iPSC DA1), or from Jejunum mucosal myofibroblasts (Lenti-iPSC JTM24) were used. Feeder-free iPSC cells were differentiated as described below.

Pancreatic differentiation was initiated by treating iPS clones with 100 ng/mL activin A (Peprotech, Rocky Hill, N.J.) and 25 ng/mL Wnt3a (R&D Systems, Minneapolis, Minn.) in advanced RPMI (A-RPMI; Invitrogen) for 1 day, followed by treatment with 100 ng/mL activin A in A-RPMI supplemented with 0.2% fetal bovine serum (FBS) (Invitrogen) for 2 days (Step 1). Differentiated cells were then cultured in A-RPMI medium containing 50 ng/mL FGF10 (R&D Systems), 0.25 μmol/L KAAD-cyclopamine (CYC), and 2% FBS for 2 days (Step 2). Next, cells were treated with 50 ng/mL FGF10, 0.25 μmol/L CYC, and 2 μmol/L all-trans retinoic acid (Sigma-Aldrich, St Louis, Mo.) in Dulbecco's modified Eagle's medium (DMEM) (Invitrogen) with 1×B27 supplement (Invitrogen) for 4 days (Step 3). Cells were then cultured in the presence of 50 ng/mL FGF10, 5 nM IndolactamV (Axxora, San Diego, Calif.), and 55 nM GLP-1 (Sigma-Aldrich) in DMEM with 1×B27 for 4 days (Step 4). Differentiation medium including 55 nmol/L GLP-1 in DMEM with 1×B27 was used to culture cells for the next 4 to 6 days (Step 5). All media were supplemented with antibiotics penicillin/streptomycin. Cells were passaged using a non-enzymatic dissociation buffer Versine at the end of steps 1, 2, 3, and 4. After step 5, cells were gently dissociated by Versine and transplanted into kidney capsules of SCID-beige mice (Charles River).

Three to 8 weeks later, almost all mice developed noticeable solid tumors/teratomas in the transplanted sites, and mice with large tumors were euthanized (FIG. 2, upper panels).

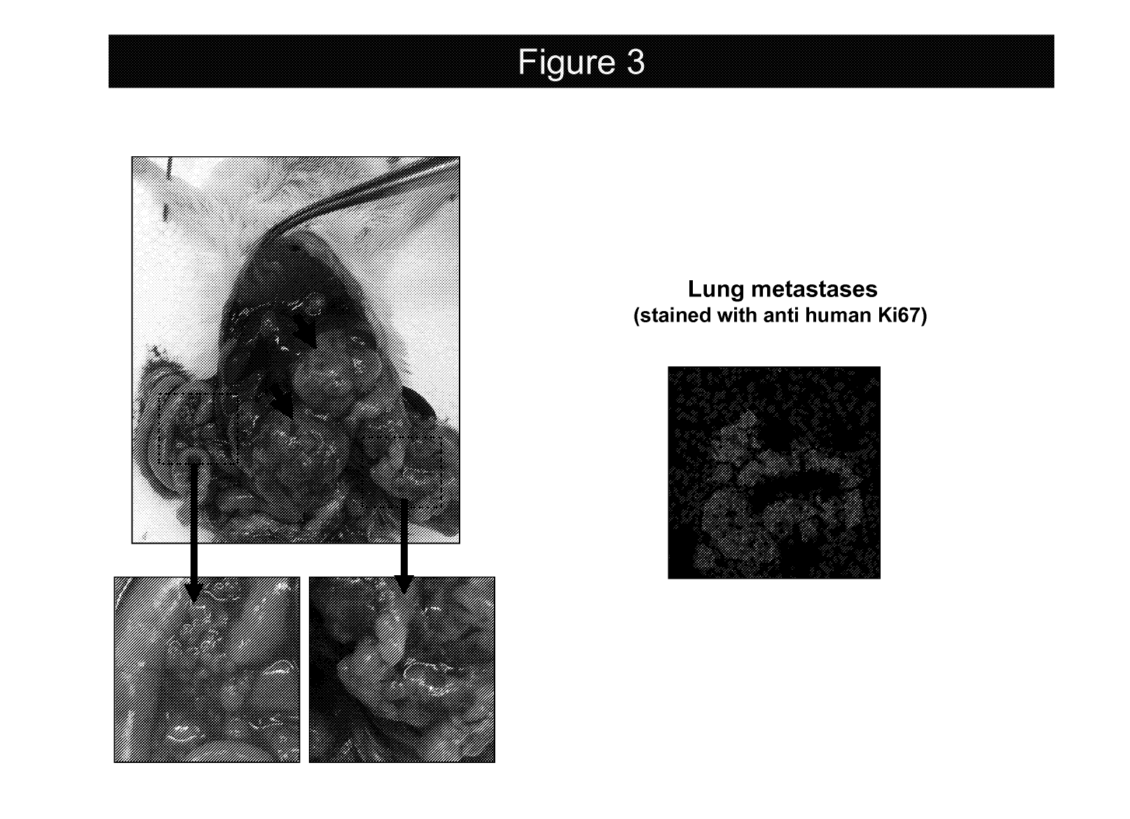

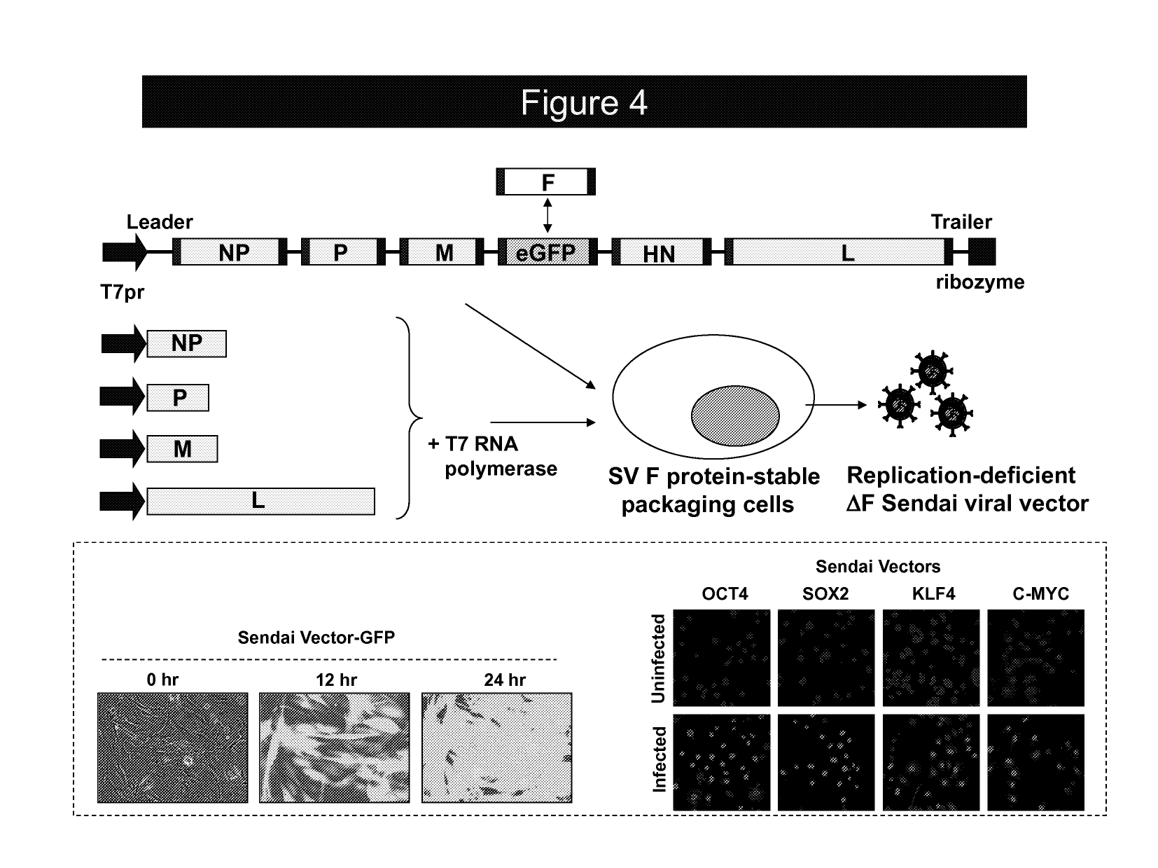

Standard unilateral nephrectomy was performed on three mice with smaller iPSC-pancreas teratomas, and one kidney with the iPSC graft/tumors was removed. Within one month, all mice with primary teratoma removal developed secondary tumors in peritoneal cavity (FIG. 3, left panel). Metastatic tumors were also found in lung or liver in two mice (FIG. 3, right panel).

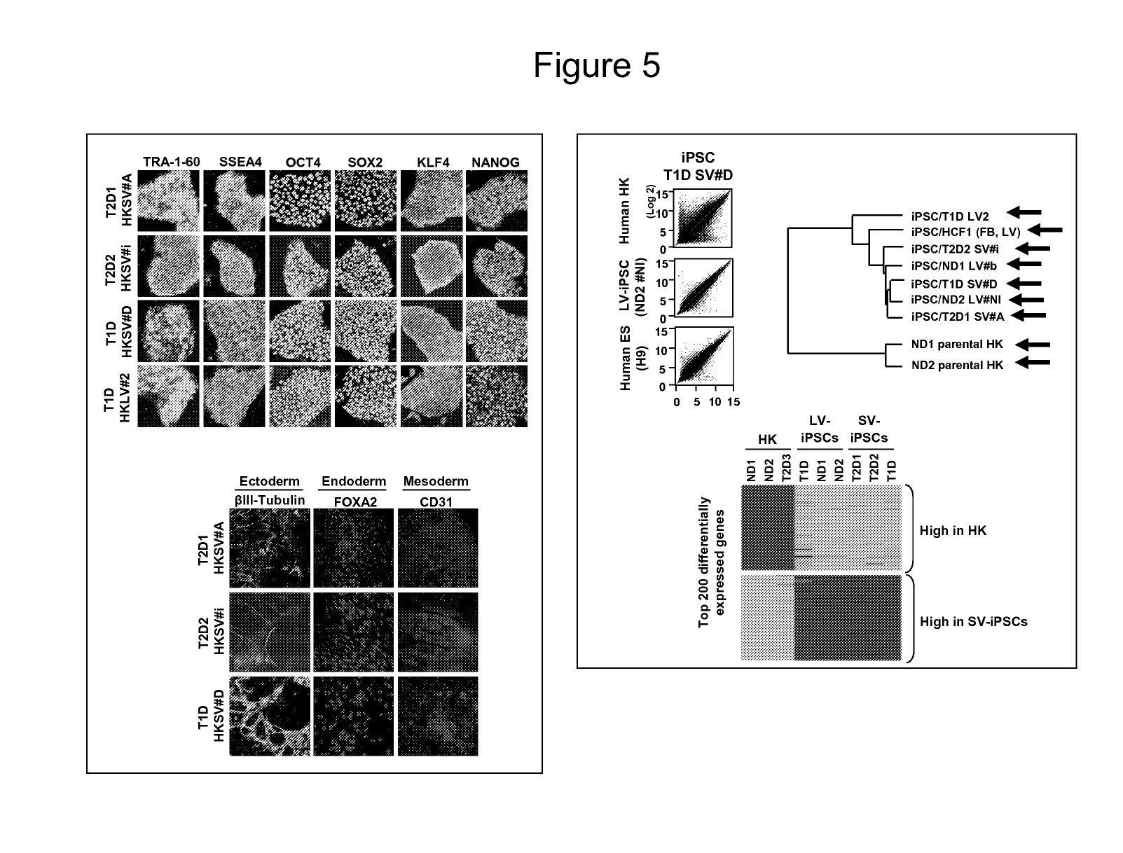

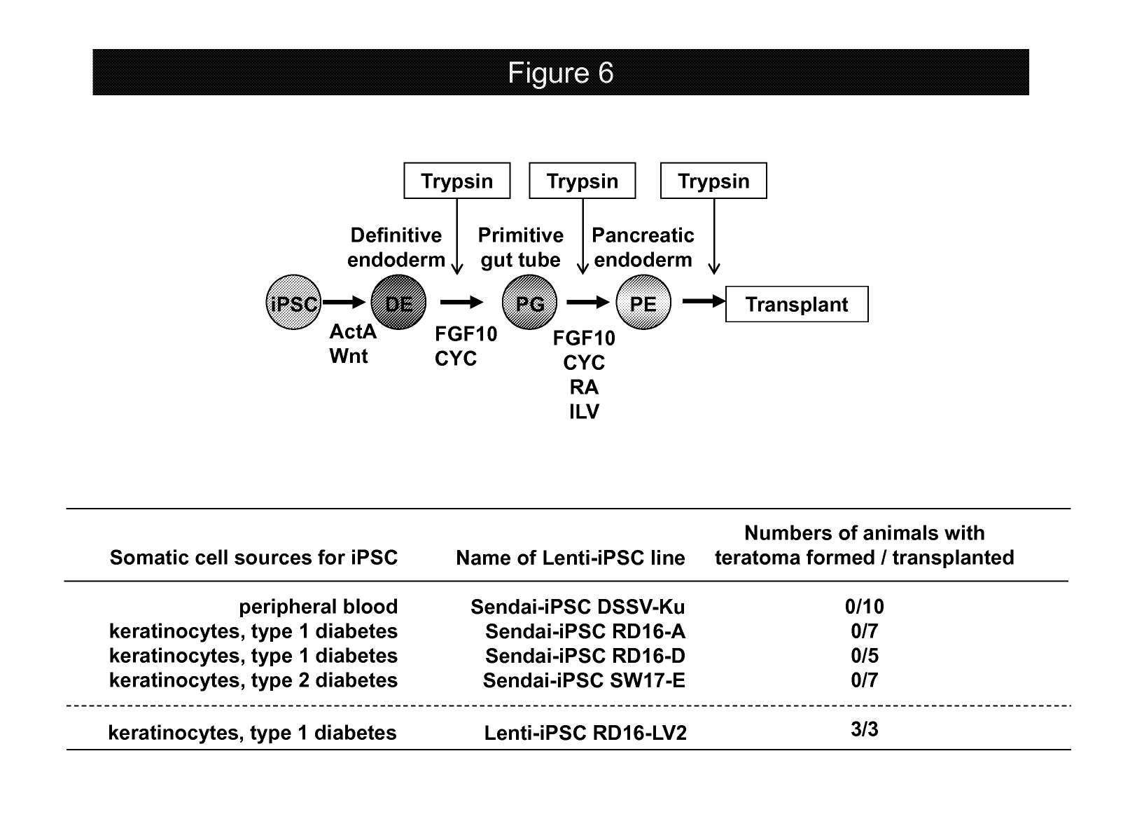

FIG. 4 describes a typical Sendai viral reprogramming vector system. Replication-defective, RNA Sendai virus-based vectors (DNAVEC, Japan) were used to express four reprogramming stemness factors (FIG. 4 lower right panels). Sendai vector-reprogrammed iPSCs (SV-iPSCs; Kudva et al., Stem Cells Translational Medicine, 1:451-461 (2012)) expressed pluripotency-associated genes (FIG. 5, upper left panels), spontaneously differentiated into three germ layers (FIG. 5, lower left panels), and showed remarkable similarities to lentivector-reprogrammed iPSCs (LV-iPSCs) by genome-wide gene expression profiling (FIG. 5, right panels).

A concern regarding the use of iPSC-derived cells is the risk of teratoma formation upon transplantation. The following was performed to test the influence of enzymatic dissociation of iPSC-derived progeny on teratoma incidence upon transplantation into immuno-compromised SCID-beige mice. For this end, Sendai vector-reprogrammed transgene-free iPSCs from blood (Sendai-iPSC DSSV-Ku) and from keratinocytes (Sendai-iPSC RD16-A, RD16-D and SW17-E) were used for pancreatic differentiation and transplantation. Lenti-iPSC RD16-LV2 cells were used as control.

Pancreatic differentiation was initiated by treating iPSC clones with 100 ng/mL activin A (Peprotech, Rocky Hill, NJ) and 25 ng/mL Wnt3a (R&D Systems, Minneapolis, Minn.) in advanced RPMI (A-RPMI; Invitrogen) for 1 day, followed by treatment with 100 ng/mL activin A in A-RPMI supplemented with 0.2% fetal bovine serum (FBS) (Invitrogen) for 2 days (Step 1). Differentiated cells were then cultured in A-RPMI medium containing 50 ng/mL FGF10 (R&D Systems), 0.25 μmol/L KAAD-cyclopamine (CYC), and 2% FBS for 2 days (Step 2). Next, cells were treated with 50 ng/mL FGF10, 0.25 μmol/L CYC, and 2 μmol/L all-trans retinoic acid (Sigma-Aldrich, St Louis, Mo.) in Dulbecco's modified Eagle's medium (DMEM) (Invitrogen) with 1×B27 supplement (Invitrogen) for 4 days (Step 3). Cells were then cultured in the presence of 50 ng/mL FGF10, 5 nM IndolactamV (Axxora, San Diego, Calif.), and 55 nM GLP-1 (Sigma-Aldrich) in DMEM with 1×B27 for 4 days (Step 4). Differentiation medium including 55 nmol/L GLP-1 in DMEM with 1×B27 was used to culture cells for the next 4 to 6 days (Step 5). All media were supplemented with antibiotics penicillin/streptomycin. Cells were passaged using a 0.25% Trypsin/EDTA solution at the end of steps 1, 2, 3, and 4. After step 5, cells were gently dissociated by a 0.25% Trypsin/EDTA solution and transplanted into kidney capsules of SCID-beige mice (Charles River).

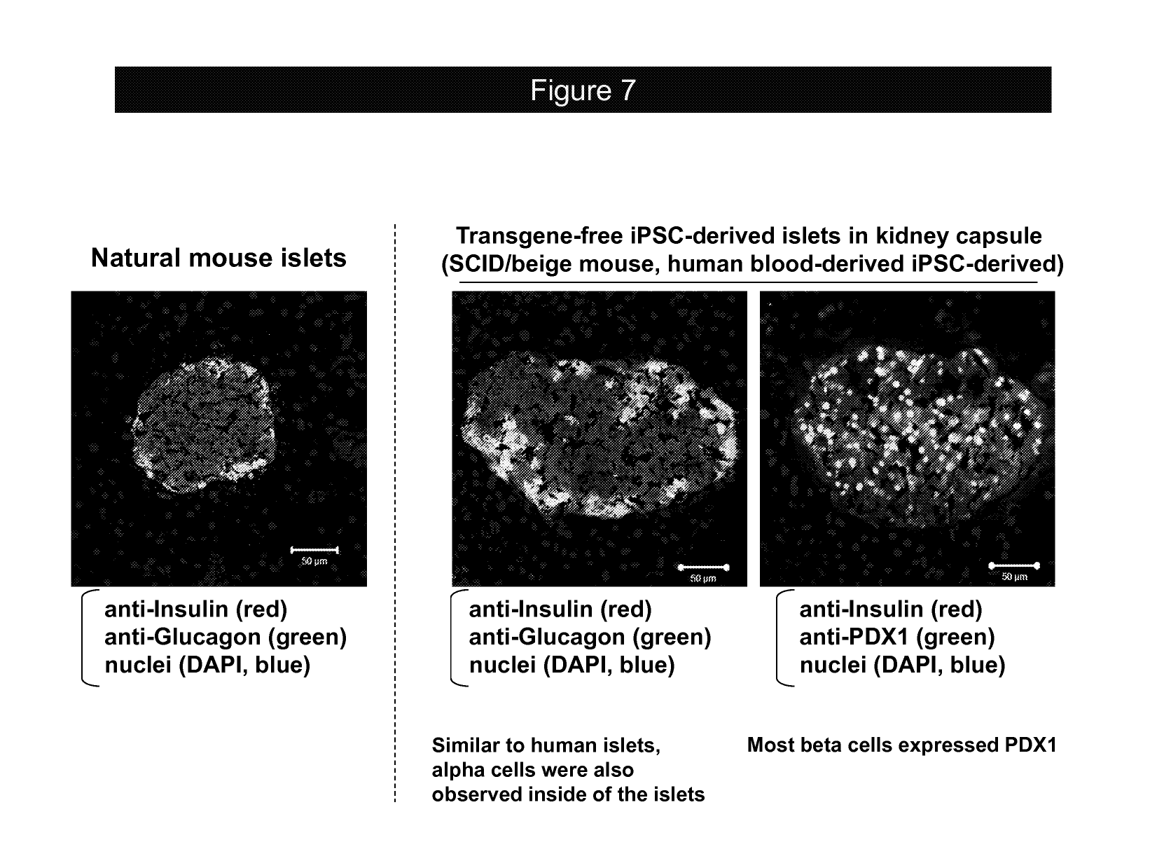

FIG. 6 showed the summary of the teratoma formation upon transplantation of iPSC-derived pancreatic endoderm cells. No teratoma formation was observed in mice transplanted with Sendai-iPSC-derived pancreatic endoderm cells. In contrast, all mice received lenti-iPSC progeny cells developed teratomas.

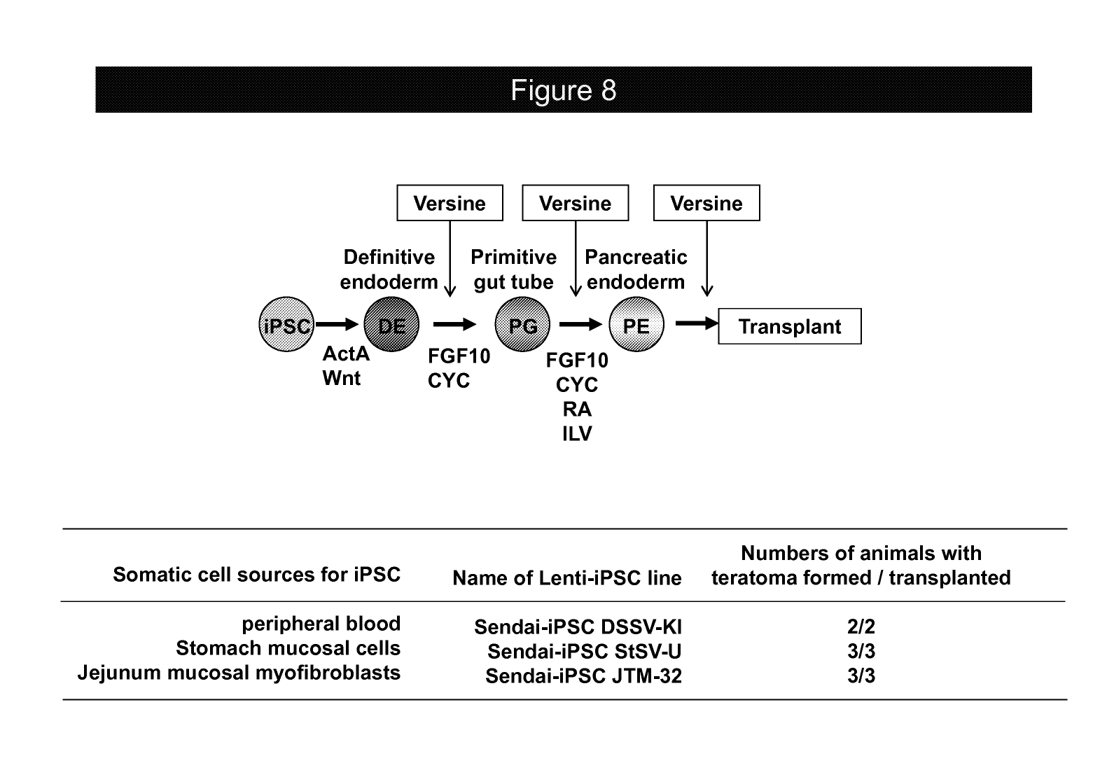

Although there were some variations, teratoma-free transplantation resulted in human islet regeneration in the kidney capsule of recipient mice (FIG. 7). iPSC-derived human islets had insulin-positive beta cells and glucagon-positive alpha cells (left panel). iPSC-derived beta cells also were positive for a mature beta cell marker, PDX1 (right panel).

To demonstrate if the enzymatic dissociation was essential for the observed teratoma-free transplantation, the same experiment was repeated using the non-enzymatic dissociation protocol (with Versine), instead of the 0.25% Trypsin/EDTA solution. As summarized in FIG. 8, all recipient mice developed teratomas, even though they received Sendai-iPSC-derived pancreatic endoderm cells.

The use of transgene-free iPSCs or the enzymatic dissociation protocol alone did not prevent tumor formation. The incidence of teratoma formation was therefore tested using transgene-free Sendai-iPSC lines, super-infected by lentiviral vectors to express the four reprogramming factors (4F), c-MYC alone or a marker GFP gene (FIG. 9, 1 of 2). Derived cells were differentiated by the Trypsin-mediated enzymatic dissociation protocol (FIG. 9, 1 of 2). As shown in FIG. 9 (2 of 2), although transplantation of Lenti-GFP-Sendai-iPSC progeny did not result in tumors, mouse recipients of Lenti-c-MYC- or Lenti-4F-Sendai-iPSC-derived cells rapidly developed teratoma/solid tumors. Thus, the use of both transgene-free iPSCs and enzymatic dissociation in iPSC differentiation steps appeared needed to achieve teratoma-free islet regeneration. In addition, c-MYC expression through an integrated reprogramming vector appeared to play a role in teratoma formation upon transplantation of iPSC progeny.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A population of differentiated cells obtained from induced pluripotent stem cells, wherein the cells of said population lack integrated viral nucleic acid encoding a stemness factor, wherein implantation of said population of differentiated cells into a mammal does not result in cancer cell formation, and wherein said cells were obtained using a culture comprising an enzyme.

2. The population of differentiated cells of claim 1, wherein said cells are human cells.

3. The population of differentiated cells of claim 1, wherein said cells lack exogenous nucleic acid.

4. The population of differentiated cells of claim 1, wherein said enzyme is trypsin.

5. A method for obtaining a population of differentiated cells obtained from induced pluripotent stem cells, wherein said method comprises:
(a) exposing somatic cells to one or more non-integrating viral vectors that direct the expression of one or more stemness factors to produce induced pluripotent stem cells from said somatic cells, and
(b) exposing said induced pluripotent stem cells to one or more differentiation factors in the presence of an enzyme to produce a population of cells more specialized than said induced pluripotent stem cells.

6. The method of claim 5, wherein said somatic cells are keratinocytes.

7. The method of claim 5, wherein said one or more non-integrating viral vector are Sendai viral vectors.

8. The method of claim 5, wherein said one or more stemness factors are an Oct3/4 polypeptide, a Sox2 polypeptide, a K1f4 polypeptide, or a c-Myc polypeptide.

9. The method of claim 5, wherein said differentiation factors are activin A, wnt3a, KAAD-cyclopamine, retinoic acid, indolactam V, IGF1, HGF, DAPT, BMP4, exendine 4, TGF-beta/BMP inhibitors, Sonic hedgehog inhibitors, PKC activators, GLP1, or a combination thereof.

10. The method of claim 5, wherein said enzyme is trypsin.
